SEQUENCE LISTING (2 sequences)

SEQ ID NO: 1 - DNA, 17 bp, Artificial Sequence
  Primer
tcccagtcacgacgttg

SEQ ID NO: 2 - DNA, 16 bp, Artificial Sequence
  Primer
aacagctatgaccatg

(19) United States
(12) Patent Application Publication
(10) Pub. No.: US 2002/0132233 A1
(43) Pub. Date: Sep. 19, 2002

(54) Development of immuno-PCR for serological diagnosis of gastric carcinoma

(76) Inventor: Jun Ren, Xian (CN)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/761,204

(22) Filed: Jan. 16, 2001

(51) Int. Cl. C12Q 1/68; G01N 33/574; G01N 33/53; C07K 16/46

(52) U.S. Cl. 435/6; 435/7.23; 435/7.5; 530/391.1

(57) ABSTRACT

Methods of detecting carcinoma-associated antigens in patient sera have been discovered. Aspects of the invention utilize single determinant immuno PCR to detect the presence or absence of a tumor associated antigens (e.g., gastric carcinoma-associated antigen MG7-Ag) in human sera. In some embodiments, a biotinylated monoclonal antibody (e.g., MG7-Ab), an avidin linker, and a biotinylated DNA are employed. The methods described herein allow for the early diagnosis of cancers, including, but not limited to, gastric carcinoma and cancers of the liver, colon, breast, uterus, and lung that display a tumor associated antigen. Some embodiments can be used to detect cancers at an early stage, to screen large populations of individuals for various cancers, diagnose the reoccurrence of cancer after surgery, and determine whether an individual suffers from metastasis.

### FIELD OF THE INVENTION

This invention concerns the detection of carcinoma-associated antigens in a biological sample. Embodiments include compositions and methods for the early diagnosis of cancer and metastasis.

### BACKGROUND OF THE INVENTION

Gastric carcinoma is one of the most significant malignancies causing morbidity and mortality in China and other Asian counties. Since gastric carcinoma is largely asymptomatic, early diagnosis is rarely possible. Only at a late stage of gastric carcinoma is this malady realized largely because the symptoms of gastric carcinoma, including vomiting, reduction of body weight, stomach ache and blood-vomiting, are brought to the attention of a wary clinician.

At the early stage of gastric carcinoma, the carcinoma cells are located at the gastric mucosa, submucosa in the inner wall of stomach. When gastric carcinoma is detected at this early stage, surgical intervention is possible and the five year survival rate can be up to 90%. Thus, early diagnosis is of great importance in improving survival.

Currently, the diagnosis of gastric carcinoma is principally based on endoscopy and X-ray based barium meal examination. Although endoscopy and pathological examination reliably diagnose gastric carcinoma, these approaches are oftentimes impractical for the screening of large populations. Further, endoscopy is an invasive and uncomfortable procedure for many, especially children and the elderly and endoscopy is impractical for evaluating the presence of carcinoma after surgery because of the threat of metastasis.

Serological testing is another approach that has been used to detect gastric carcinoma. Presently, several commercial kits for the serodiagnosis of gastric carcinoma are available. These kits employ antibodies directed to tumor-associated antigens (TAA) such as carcinoembryonic antigen (CEA), CA 50, and CA 19-9. Unfortunately, the level of serum TAA is frequently undetectable when routine immunoassays are employed, particularly in patients with early tumors. In fact, commercial kits for the serodiagnosis of gastric carcinoma (e.g., CEA, CA 50, and CA 19-9) show a diagnosis rate for the detection of early stage gastric carcinoma at about 40%-50%. For this reason, the serological diagnosis method is largely impractical for the early clinical diagnosis of gastric carcinoma.

A more sensitive antigen detection system, termed immunopolymerase chain reaction (immuno-PCR), has been developed recently. This method combines PCR and enzyme linked immunoabsorbent assay (ELISA), using a specifically designed chimeric protein that has a bispecific binding affinity for DNA and antibodies that serve as a linker molecule. In 1984, this technique was used to detect a gastric carcinoma-associated antigen in tissue culture cells (1). Although promising, a key component in the technique, a streptavidin-protein A fusion protein, has a widely varied affinity for antibodies of various classes and subclasses, thus, clinical application of the technique was not feasible. In view of the foregoing, and notwithstanding the various efforts exemplified in the prior art, there remains a need for new approaches to diagnose gastric carcinoma.

### BRIEF SUMMARY OF THE INVENTION

A very sensitive method of detecting carcinoma-associated antigens in patient sera has been discovered. Aspects of the invention utilize single determinant immuno-PCR to detect the presence or absence of a tumor associated antigens, “TAA”, (e.g., gastric carcinoma-associated antigen MG7-Ag) in human sera. In some embodiments, a biotinylated monoclonal antibody (e.g., MG7-Ab), an avidin linker, and a biotinylated DNA (referred to as a “diagnostic reagent”) are employed. The methods described herein allow for the early diagnosis of cancers, including, but not limited to, gastric carcinoma and cancers of the liver, colon, breast, uterus, and lung that display a TAA. Some embodiments can be used to detect cancers at an early stage, to screen large populations of individuals for various cancers, diagnose the reoccurrence of cancer after surgery, and determine whether an individual suffers from metastasis.

Embodiments include a diagnostic reagent comprising an MG7 antibody or fragment thereof joined to an avidin linker joined to a DNA having at least 50 nucleotides in length. In some embodiments the DNA is at least 200 or at least 500 nucleotides in length. In one embodiment, in particular, the DNA is a plasmid DNA such as pXJ 19. Further, the avidin linker can be free-avidin or avidin joined to a support such as, an avidin resin. Additionally, an embodiment of the invention includes a biological complex comprising the diagnostic reagent described above joined to a tumor associated antigen (TAA).

Methods of the invention include a method of identifying the presence or absence of a tumor associated antigen (TAA) in a biological sample from an animal. By one approach, this method is practiced by obtaining a biological sample from the animal; contacting the biological sample with a biotinylated antibody that specifically detects the tumor associated antigen so as to form an antibody/antigen complex; contacting the antigen/antibody complex with an avidin linker so as to form an antigen/antibody/avidin linker complex; contacting the antigen/antibody/avidin linker complex with a biotinylated DNA so as to form an antigen/antibody/avidin linker/DNA complex; performing a Polymerase Chain Reaction (PCR) in the presence of the antigen/antibody/avidin linker/DNA complex; and detecting the presence or absence of a PCR extension product that corresponds to the biotinylated DNA whereby the presence or absence of the TAA in the biological sample is identified. In some methods, the TAA is associated with gastric carcinoma or with a cancer selected from the group consisting of esophageal carcinoma, colon carcinoma, liver carcinoma, ovary carcinoma, uterine carcinoma, and lung carcinoma. Further, in many methods, the antibody is MG7 or the DNA is pXJ 19 or the animal is a human or the biological sample is human sera.

By another approach, the method of identifying the presence or absence of a tumor associated antigen (TAA) in a biological sample from an animal is practiced by obtaining a biological sample from the animal; contacting the biological sample with a diagnostic reagent comprising an antibody that specifically detects the tumor associated antigen, an avidin linker, and a biotinylated DNA so as form an antigen/diagnostic reagent complex; performing a Polymerase Chain Reaction (PCR) in the presence of the antigen/diagnostic reagent complex; and detecting the presence or absence of a PCR extension product that corresponds to the biotinylated DNA whereby the presence or absence of the TAA in the biological sample is identified. As above, aspects of this embodiment have a TAA that is associated with gastric carcinoma or with a cancer S selected from the group consisting of esophageal carcinoma, colon carcinoma, liver carcinoma, ovary carcinoma, uterine carcinoma, and lung carcinoma. Additionally, this embodiments can include the MG7 antibody or the pXJ 19 DNA and the animal can be human and the biological sample can be human sera.

Another method is concerned with the identification of the presence or absence of metastasis in an animal. This method is practiced by obtaining a biological sample from the animal; contacting the biological sample with a biotinylated antibody that specifically detects a tumor associated antigen (TAA) so as to form an antibody/antigen complex; contacting the antigen/antibody complex with an avidin linker so as to form an antigen/antibody/avidin linker complex; contacting the antigen/antibody/avidin linker complex with a biotinylated DNA so as to form an antigen/antibody/avidin linker/DNA complex; performing a Polymerase Chain Reaction (PCR) in the presence of the antigen/antibody/avidin linker/DNA complex; determining the amount of PCR extension product that corresponds to the biotinylated DNA, whereby the amount of TAA in the biological sample is determined; and comparing the amount of TAA determined in step the above with a standard, the amount of TAA in a biological sample obtained from an animal that has metastasis or the amount of TAA in a biological sample obtained form an animal that has cancer but does not have metastasis, whereby the presence or absence of metastasis in the animal is identified. In this method, the antibody can be MG7, the DNA can be pXJ 19, the animal can be a human and the biological sample can be human sera.

In still another embodiment, a method of identifying the presence or absence of metastasis in an animal is provided. This method is practiced by obtaining a biological sample from the animal; contacting the biological sample with a diagnostic reagent comprising a biotinylated antibody that specifically detects a tumor associated antigen (TAA), an avidin linker, and a biotinylated DNA so as to form an antigen/diagnostic reagent complex; performing a Polymerase Chain Reaction (PCR) in the presence of the antigen/diagnostic reagent complex; determining the amount of PCR extension product that corresponds to the biotinylated DNA, whereby the amount of TAA in the biological sample is determined; and comparing the amount of TAA determined in the step above with a standard, the amount of TAA in a biological sample obtained from an animal that has metastasis or the amount of TAA in a biological sample obtained form an animal that has cancer but does not have metastasis, whereby the presence or absence of metastasis in the animal is identified. In this method, the antibody can be MG7, the DNA can be pXJ 19, the animal can be a human and the biological sample can be human sera.

Other embodiments include a kit comprising the diagnostic reagents described above. Some aspects of the kit embodiments include instructions and/or the primers of SEQ. ID. No. 1 and 2.

### DETAILED DESCRIPTION OF THE INVENTION

The invention described herein is concerned with the diagnosis of cancers in a biological sample obtained from an animal, preferably human sera. One embodiment involves a sensitive method for detecting the presence or absence of a carcinoma-associated antigen in animal sera. This embodiment allows for the early diagnosis of cancers. Other embodiments involve the screening of patients for the presence of metastasis or the reoccurrence of cancer after a cancer therapy (e.g., chemotherapy, radiation, antibody therapy, or surgery).

One embodiment uses single determinant immuno-PCR to detect the presence of the gastric carcinoma-associated antigen MG7-Ag in the human sera of gastric carcinoma patients. In this embodiment, a novel detection reagent comprising a biotinylated monoclonal MG7-Ab joined to an avidin linker and a biotinylated DNA is employed. Once the antibody is attached to an antigen, the Polymerase Chain Reaction (PCR), using primers specific for the biotinylated DNA, is initiated and detection of the presence of the antigen, as well as the quantity of the antigen in the sample, can be determined by conventional means of PCR analysis (e.g., gel electrophoresis and ethidium bromide staining). Additionally, by conducting the PCR reaction in the presence of a radiolabeled deoxyribonucleotide triphosphate (e.g., P³² dCTP) or a radiolabeled primer, one of skill in the art can detect the presence or absence of the antigen, as well as the quantity of the antigen detected, using gel electrophoresis and autoradiography. These novel approaches allow for the detection of as few as 20 cells, providing approximately a 10,000-fold enhancement in sensitivity of detection, when compared to conventional ELISA. The section below describes the preparation of the diagnostic reagent used with many of the embodiments described herein.

Preparation of the Diagnostic Reagent

One embodiment of the diagnostic reagent comprises a biotinylated antibody (or fragment thereof) that interacts with a TAA joined to an avidin linker that is joined to a biotinylated DNA, which serves as a template for PCR. It is important to note that a diagnostic reagent can comprise many different antibodies or fragments thereof, many different types of avidin linkers or avidin-like linkers (e.g., avidin, streptavidin, neutravidin and other molecules that bind biotin), and many different biotinylated DNAs. The next section describes the manufacture of antibodies for use with the diagnostic reagents described herein.

Preparation of Antibodies Directed to a TAA

Depending on the context, the term “antibodies” can encompass polyclonal, monoclonal, chimeric, single chain, Fab fragments and fragments produced by a Fab expression library. Preferably these antibodies used with aspects of the invention are monoclonal antibodies. For the production of antibodies, various hosts including goats, rabbits, rats, mice, etc. can be immunized by injection with the TAA or any portion, fragment or oligopeptide that retains immunogenic properties. Depending on the host species, various adjuvants can be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. BCG (Bacillus Calmette-Guerin) and Corynebacterium parvum are also potentially useful adjuvants.

Peptides used to induce specific antibodies can have an amino acid sequence consisting of at least three amino acids, and preferably at least 10 to 15 amino acids. Preferably, short stretches of amino acids encoding fragments of a TAA are fused with those of another protein such as keyhole limpet hemocyanin such that an antibody is produced against the chimeric molecule. While antibodies capable of specifically recognizing a TAA can be generated by injecting synthetic 3-mer, 10-mer, and 15-mer peptides that correspond to a protein sequence of a TAA into mice, a more diverse set of antibodies can be generated by using a recombinant TAA, purified TAA, or fragments of TAA.

To generate antibodies to a TAA and fragments of a TAA, substantially pure TAA or a fragment of TAA is isolated from a transfected or transformed cell. The concentration of the polypeptide in the final preparation is adjusted, for example, by concentration on an Amicon filter device, to the level of a few micrograms/ml. Monoclonal or polyclonal antibody to the polypeptide of interest can then be prepared as follows:

Monoclonal antibodies to TAA or a fragment of TAA can be prepared using any technique that provides for the production of antibody molecules by continuous cell lines in culture. These include, but are not limited to, the hybridoma technique originally described by Koehler and Milstein (Nature 256:495-497 (1975), the human B-cell hybridoma technique (Kosbor et al. Immunol Today 4:72 (1983); Cote et al Proc Natl Acad Sci 80:2026-2030 (1983), and the EBV-hybridoma technique Cole et al. Monoclonal Antibodies and Cancer Therapy, Alan R. Liss Inc, New York N.Y., pp 77-96 (1985). In addition, techniques developed for the production of “chimeric antibodies”, the splicing of mouse antibody genes to human antibody genes to obtain a molecule with appropriate antigen specificity and biological activity can be used. (Morrison et al. Proc Natl Acad Sci 81:6851-6855 (1984); Neuberger et al. Nature 312:604-608(1984); Takeda et al. Nature 314:452-454(1985). Alternatively, techniques described for the production of single chain antibodies (U.S. Pat. No. 4,946,778) can be adapted to produce TAA-specific single chain antibodies. Antibodies can also be produced by inducing in vivo production in the lymphocyte population or by screening recombinant immunoglobulin libraries or panels of highly specific binding reagents as disclosed in Orlandi et al., Proc Natl Acad Sci 86: 3833-3837 (1989), and Winter G. and Milstein C; Nature 349:293-299 (1991).

Antibody fragments that contain specific binding sites for TAA can also be generated. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries can be constructed to allow rapid and easy identification of monoclonal Fab fragments with the desired specificity. (Huse W. D. et al. Science 256:1275-1281 (1989)).

By one approach, monoclonal antibodies to TAA or fragments thereof are made as follows. Briefly, a mouse is repetitively inoculated with a few micrograms of the selected protein or peptides derived therefrom over a period of a few weeks. The mouse is then sacrificed, and the antibody producing cells of the spleen isolated. The spleen cells are fused in the presence of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol. 70:419 (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, N.Y. Section 21-2.

Polyclonal antiserum containing antibodies to heterogenous epitopes of a single protein can be prepared by immunizing suitable animals with the expressed protein or peptides derived therefrom described above, which can be unmodified or modified to enhance immunogenicity. Effective polyclonal antibody production is affected by many factors related both to the antigen and the host species. For example, small molecules tend to be less immunogenic than others and can require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J. Clin. Endocrinol. Metab. 33:988-991 (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980). Preferably, polyclonal antibodies are purified by precipitation and/or affinity chromatography.

Once the antibody (monoclonal or polyclonal) or fragment thereof is prepared it is biotinylated. There are many ways of biotinylating antibodies for use with the diagnostic reagents described herein (e.g., NHS-biotin, photobiotin, and biotinylated secondary antibodies). The use of photobiotin is the preferred approach, however. Example 1 describes an approach that was used to make a biotinylated anti-MG7 antibody.

### EXAMPLE 1

### Preparation of a Biotinylated Monoclonal Antibody

A hybridoma cell line producing the monoclonal antibody (mAb) MG7 directed against the gastric carcinoma-associated antigen was developed as follows. The hybridoma cells were isolated from DMEM (Dulbecco's Modified Eagle's Medium) or RPMI 1640, and washed with Phosphate Buffered Saline (PBS) to remove albumin. These cells were injected to mice i.p. to produce the abdominal dropsy. The sera were collected by separating out red blood cells and saturated ammonium sulfate was added. The mixtures were centrifuged and were washed with PBS. The antibody was purified by DEAE-52 cellulose affinity chromatography.

The in vitro biotinylation of mAbs was accomplished using photobiotin. Briefly, a suspension of the mAb in PBS was mixed with the photbiotin in aqueous solution (the molar ratio of antibody to photobiotin was 50). The tube was placed in an ice bath, held approximately 10 cm under a sunlamp, and illuminated for 15 minutes. The biotinylated mAbs were then purified on a sepharose G-50 minicolumn, according to the manufacturer's instructions. The next section describes the manufacture of the biotinylated DNA for use with the novel diagnostic reagent.

Preparation of a Biotinylated DNA for Use as a Template for PCR

The DNA that is used for the biotinylated DNA template can be virtually any DNA that is capable of being amplified by PCR. In this respect, the DNA serves as a marker for detection. Preferably, the biotinylated DNA template is sufficiently long to produce a PCR product that is easily detectable (e.g., a PCR product of greater that 200 bp) and the placement of the biotin does not interfere with PCR amplification. That is, some embodiments have a DNA that is at least 50, 60, 70, 80, 90, 100, 125, 150, 175, 200, 250, 300, 350, 400, 450, 500, 550, 600, 650, 700, 750, 800, or 850 or more nucleotides in length. In some embodiments, biotinylated oligonucleotides are used. The incorporation of biotin can be accomplished during synthesis of the oligonucleotides such that biotin molecules are selectively incorporated at specific regions of the oligonucleotide that do not interfere with PCR amplification (e.g., at the 5′ or 3′ end of the oligonucleotide). Plasmid DNA that is biotinylated through the use of photobiotin can also be used. The plasmid DNA can remain circular or can be linearized by cleavage with a restriction endonuclease. Example 2 describes the preparation of a biotinylated plasmid DNA for use with a diagnostic reagent.

### EXAMPLE 2

Preparation of a Biotinylated Plasmid DNA

In some embodiments, the full-length recombinant plasmid, pXJ 19, was biotinylated and used to construct the diagnostic reagent. Approximately, 0.34 μg/μl of the pXJ 19 plasmid was labeled with photobiotin acetate (1 μg/μl) in a tube that was placed in an ice bath by subjecting the tube to illumination by a sunlamp (10 cm from the tube) for approximately 15 minutes. Once the photo reaction was complete 2-butanol was added and the plasmid was precipitated by chilling and centrifigation. The pellet was resuspended in a suitable buffer and prepared for conjugation with the avidin linker. The next section describes conjugation of biotinylated antibody and biotinylated DNA with the avidin linker.

Preparation of the Avidin Linker and Establishment of Standards

Embodiments of the invention utilize avidin or an avidin-like material that can be free (i.e., without attachment to a support) or immobilized (e.g., joined to a support, carrier, or resin). Thus, for some applications, e.g., embodiments that use an avidin resin, it may be necessary to block non-specific binding prior to adding the biotinylated antibody and/or biotinylated DNA. There are many blocking solutions known in the art and a solution of bovine serum albumin or dry milk with tRNA or carrier RNA is suitable. Preferably, the avidin resin is blocked overnight on a rocker in a cold room. Once the avidin resin is sufficiently blocked the resin is washed in PBS to remove the blocking solution. Next, the biotinylated antibody and/or biotinylated DNA can be added.

In some embodiments, it is desired that the antibody is joined to the avidin (or avidin resin, collectivelly referred to as the “avidin linker”) prior to the DNA, however, with other embodiments it may be necessary to join the DNA prior to joining the antibody. In still other embodiments, the biotinylated antibody and biotinylated DNA are added to the avidin linker simultaneously. In still more embodiments, the biotinylated antibody is first added to the antigen-containing sample, then the avidin linker is added, and, finally, the biotinylated DNA is added. Preferably, washing steps with PBS are employed to remove unbound antibody and/or DNA during the incorporation steps described above and after both the antibody and the DNA have been joined to the avidin linker.

Although the conditions for binding an avidin linker can be adjusted such that nearly 100% of the avidin linker is joined by at least one biotinylated antibody and one biotinylated DNA, it may be desired to quantify the incorporation of antibody and/or DNA with the avidin linker. By using an iodinated antibody and a P³² labeled DNA, one of skill in the art can rapidly determine the amount of antibody and DNA bound to a given volume of avidin linker. For example, the biotinylated antibody can be iodinated using conventional techniques (Pierce Chemical) and the biotinylated DNA can be labeled with P³² using nick translation or T4 kinase end-labeling. Once the biotinylated antibody and DNA are radiolabeled, they are bound to the avidin linker, as described above. The avidin linker is washed in PBS to remove any unbound antibody or DNA and various amounts of the diagnostic reagent are then placed in scintillation vials. The amount of the two types of radioactivity are then determined by scintillation counting. If the specific activity of the iodinated antibody and the P³² labeled DNA are known, the amount of antibody and DNA for a given volume of diagnostic reagent can be calculated.

In some cases, it is also desirable to quantify the immuno-PCR reaction. This can be accomplished by attaching a known concentration of antigen that interacts with the antibody on the diagnostic reagent to a support (e.g., a nylon or nitrocellulose membrane or, preferably, a PCR reaction tube) and performing immuno-PCR, as described herein. Preferably, the amount of antigen that is attached to the support and the amount of diagnostic reagent added to the antigen is titrated over several samples. For example, one group of tubes has an identical amount of antigen bound and the amount of diagnostic reagent added to the tubes is titrated and in a second group of tubes, the amount of antigen bound to the tubes is titrated and the amount of diagnostic reagent added to the tubes is titrated. By titrating various amounts of antigen with various amounts of diagnostic reagent one can create an array allowing for a fine quantification of PCR ampplification.

After washing away the un-affixed diagnostic reagent, the two groups of tubes are subjected to PCR amplification, as described below. The conditions for PCR are kept constant and, preferably, a P³² labeled primer (kinase end-labeled) is used so as to create radioactive extension products. Preferably, the specific activity of the P³² labeled primer is determined so that the amount of extension product can be calculated. The radiolabeled extension products are isolated from the reaction and are separated on a polyacrylamide gel. The gel is subjected to autoradiography and can be quantified by visually inspecting the bands or by using a densitometer. The radioactivity can also be quantified by removing the radioactive band and performing liquid scintillation counting.

Accordingly, one of skill in the art can determine the amount of extension product that is generated from various amounts of diagnostic reagent (having a known amount of antibody and DNA, as determined above) and various amounts of antigen. This information can be plotted and used as a standard for comparison and quantification of the amount of antigen detected in clinical samples can be accomplished. The nest section describes the manufacture of kits having diagnostic reagents or components thereof.

Kits Having the Diagnostic Reagents

Additional embodiments include the preparation of diagnostic kits comprising the diagnostic reagents or components thereof. The diagnostic reagents or components thereof (e.g., biotinylated antibody, such as MG7, avidin, and/or biotinylated DNA) will typically be supplied in combination with one or more of the following reagents. A support capable of absorbing or otherwise binding antigen. Available supports for this purpose include, but are not limited to, membranes of nitrocellulose, nylon or derivatized nylon that can be characterized by bearing an array of positively charged substituents, and Genechips™ or their equivalents. One or more enzymes, such as Taq polymerase, can be furnished in the kit, as can dNTPs, buffers, and instructions that teach how to perform the methods described herein. Results from the kit assays can be interpreted by a healthcare provider or a diagnostic laboratory. Alternatively, diagnostic kits are manufactured and sold to private individuals for self-diagnosis. The next section describes the preparation of clinical samples for the immuno-PCR technique described herein.

Sample Preparation

The types of biological samples that can be used with the embodiments described herein are vast and limited only by the presence of a TAA. Preferably, human sera is used. In one embodiment, the sera tested in the immuno-PCR was obtained from patients who had unresectable gastric, colorectal, esophageal, liver, ovarian, uterine, or lung carcinomas with and without metastasis. Tumor tissue blocks were also resectioned for confirmation of histological diagnosis and histochemical staining. The example below describes the preparation of sera and the types of sera that were used to verify the embodiments described herein.

### EXAMPLE 3

### Sera Preparation

All sera were collected prior to surgery unless otherwise stated. Sera samples were collected from blood donors and patients with carcinoma including gastric carcinoma (198 cases), esophageal carcinoma (86 cases), colonic carcinoma, (90 cases), liver carcinoma (84 cases), ovarian carcinoma (45 cases), uterine carcinoma (27 cases), lung carcinoma (66 cases), as well as from patients with benign diseases including peptic ulcer (78 cases) chronic gastritis (118 cases), and chronic colitis (60 cases); sera also were collected from 236 healthy blood donors. All samples were frozen and stored at −20° C. until used. Aliquots of serum samples were diluted three times in PBS with 1% bovine serum albumin (BSA) prior to immuno-PCR assay. The next section describes the immuno-PCR technique in greater detail.

Immuno-PCR for Tumor Associated Antigens

The single determinant immuno-PCR can be performed by many different techniques. For example, sera can be used to coat a number of different supports (e.g., PCR tubes, nitrocellulose or nylon filters on a dot-blot manifold) and the immuno-PCR reaction can be performed directly on the support, if a coated vessel is used, or indirectly by cutting out the portion of the membrane containing the bound sera, placing it into a PCR tube. The example below describes another approach that was used to conduct immuno-PCR on sera obtained from several different patients.

### EXAMPLE 4

### Immuno-PCR with MG7 Antibody/pXJ 19 Diagnostic Reagent

The single determinant immuno-PCR assay was conducted as follows. Microtiter wells containing 000 μL of diluted serum was coated with 0.05M carbonate buffer (pH 9.6) at 4° C. overnight or at 37° C. for 2 hours. Wells were washed twice with PBS-Tween 20 and blocked(180 μL/well) with 3.0% nonfat dry milk, 0.1 mM ethylenediamine tetraaccetic acid, and 0.02% sodium azide in PBS. After washing 3 times, 100 μL of biotinylated MG7 antibody (10 μg/mL) was added and incubated at 37° C. for 2 hours. Continuously washing steps were followed to remove the unbound antibody, a slight excess of avidin (7.2 μg/mL) was added to bind attached biotinylated MG7 and incubated at room temperature for 30 minutes. Subsequently, the biotinylated pXJ 19 DNA was added to the complex for an additional incubation under the same conditions. The wells were washed with Tween-PBS for 5 minutes, 5 times and with distilled water for 5 minutes, 4 times to remove nonspecifically bound biotinylated plasmid DNA. Approximately 50 μL of PCR mixture was added to the wells and a denaturation step of 3 minutes at 96° C. was performed within the wells to allow the bound biotinylated plasmid pXJ 19 to detach. The content of each well then was transferred into a 500 μL Eppendorf tube. Each tube was subjected to PCR using a DNA thermal cycler (Perkin Elmer Cetus, Norwalk, Conn.). Nucleotide sequences of the primers were 5′-TCCCAGTCACGACGTTG-3′ SEQ. ID. No. 1 for the sense direction and 5′-AACAGCTATGACCATG-3′ SEQ. ID. No. 2 for the antisense direction.

PCR was performed under the following conditions: 10 mM Tris-HCl (pH 8.3), 50 mM KCl, 1.5 mM MgCl2, 0.8 mM deoxyribonucleotide (0.2 mM each), 2M of each primer, and 1.25 units of Tag DNA polymerase. The temperature profile used was an initial denaturation at 96° C., for 3 minutes, 35 cycles of denaturation (at 94° C. for 1 minute), annealing (at 40° C. for 1 minute), and extension (at 72° C. for 1 minute), with final extension at 72° C. for 5 minutes. The primers generate a 420 bp extension product. Ten microliters of the resulting PCR products were separated on a 2% agarose gel electrophoresis and were stained with ethidium bromide. The presence of the 420 bp DNA fragment indicated that the sera sample had TAA that was positive for gastric carcinoma. The results of these experiments are provided in TABLE 1.

TABLE 1
MG7-Ag Detection rate by Immuno-PCR in the Sera of Patients
with Gastric Carcinoma and Other Diseases.

| Positive Group | No. of cases | No. of positive | % positive |
|---|---|---|---|
| Gastric Ca. | 198 | 164 | 82.8 |
| Esophageal Ca. | 86 | 15 | 17.4 |
| Colon Ca. | 90 | 40 | 44.4 |
| Liver Ca. | 84 | 0 | 0.0 |
| Ovarian Ca. | 45 | 1 | 2.2 |
| Uterine Ca. | 27 | 0 | 0.0 |
| Lung Ca. | 66 | 4 | 6.1 |
| Peptic Ulcer | 78 | 6 | 7.7 |
| Chronic Gastritis | 118 | 7 | 5.9 |
| Chronic Colitis | 60 | 2 | 3.3 |
| Blood Donor | 236 | 2 | 0.8 |

The gastric carcinoma-associated antigen MG7-Ag was detected by Immuno-PCR in 82.8% of serum samples of patients with gastric carcinoma (164 of 198). A cross reaction with other TAAs is suspected, since positive results were detected in the patients of other carcinomas of the gastrointestinal tract such as esophageal carcinoma and colon carcinoma etc. Thus, the MG7 antibody/pXJ 19 diagnostic reagent can be used to detect the presence or absence of gastric carcinoma, esophageal carcinoma, and colon carcinoma. The next example demonstrates that the inventive immuno-PCR method described herein is significantly more sensitive than currently available detection approaches.

### EXAMPLE 5

### Comparative Study of Diagnosis Rate on Immuno-PCR for Mg7-Ag and Commercial Kits for TAA.

A comparison of the efficacy of currently available diagnostic approaches with the methods of the invention was also performed. Accordingly, RIA for CEA, CA 50, CA19-9, and TAG-72, IRMA for MG7-Ag, and immuno-PCR for MG7-Ag were performed on the samples that are listed in TABLE 2. The experiment was performed on 86 gastric carcinoma patients and 83 benign disease patients. Seventy of 86 gastric carcinoma patients were seropositive for MG7-Ag by Immuno-PCR, whereas only 24 and 42 patients, respectively (27.9% and 48.4%, respectively), were positive by the other methods. The sensitivity and specificity of the current invention were 81.4%, and 91.6%, respectively, which is much more accurate than other method tested.

TABLE 2
Comparative study of immuno-PCR Assay for MG7-Ag with CEA,
CA 50, CA 19-9, TAG-72, and MG7-Ag IRMA

| | Gastric | | | |
|---|---|---|---|---|
| | Carcinoma | | Benign | |
| Antigen | (n = 86) | % | (n = 83) | % |
| CEA | 29 | 33.7 | 10 | 12.0 |
| CA 50 | 24 | 27.9 | 6 | 7.2 |
| CA 19-9 | 33 | 38.4 | 6 | 7.2 |
| TAG-72 | 39 | 45.3 | 7 | 8.4 |
| MG-Ag IRMA | 42 | 48.8 | 6 | 7.2 |
| MG-Ag IPCR | 70 | 81.4 | 7 | 8.4 |

PCR: polymerase chain reaction;
CEA: carcinoembryonic antigen
IRMA: immunoradiometric assay;
IPCR: immuno-polymerase chain reaction

The next section describes methods of using the embodiments described herein to identify a patient having metastasis.

Identification of Metastasis Using a MG7 Antibody/pXJ 19 Diagnostic Reagent

Other embodiments of the invention concern the identification of individuals suffering from metastasis. Because a greater quantity of the MG7 antibody/pXJ 19 diagnostic reagent associates with individuals suffering from metastasis than individuals that do not have metastasis it was discovered that this diagnostic reagent can be used to identify individuals suffering from metastasis. By one approach, a profile is initially established. Preferably, several individuals suffering from non-metastic gastric carcinoma, for example, are screened and the amount of antigen in these patients is determined by comparing the amount of extension products generated to the standard array described above. These values are plotted and a baseline range for non-metastic gastric carcinoma is established. A similar process can be performed for many other types of non-metastic cancer so that library of multiple baselines can be created for comparison to samples for identification.

Additionally, it is desirable to create a baseline for metastic cancers. Again, multiple patients suffering from metastic cancer are analyzed, the amount of antigen is quantified and these results are plotted so as to generate a baseline. As above, this process can be repeated with many other types of metastic cancer so as to generate a library of multiple baselines for comparison to samples for identification. The results from patients suffering from metastic cancer can then be coordinated with the results from patients suffering from non-metastic cancer so as to have a better profile for comparison to the levels of antigen detected in a screened sample. By comparing the levels of antigen detected in a screened sample to a profile comprising the ranges of acceptable values for both metastic and non-metastic cancers, one of skill in the art can rapidly determine the likelihood that the screened individual suffers from metastasis. The example below describes another approach that was used to determine whether or not a patient suffers from gastric carcinoma that is accompanied by metastasis.

### EXAMPLE 6

### Semiquantitative Analysis of PCR Products from Patients with Gastric Carcinoma with and without Metastasis

Quantitation of the PCR products generated from serum samples obtained from patients suffering from gastric carcinoma (with or without metastasis) was determined by separating the amplified DNA on agarose gels, staining with ethidium bromide, and scanning. It was found that band density was significantly greater in samples from patients with metastasis, indicating that the concentration of MG7-Ag in the serum of those patients was higher, than in patients without metastatic disease. (See TABLE 3). Accordingly, these results provide evidence that the MG7/pXJ 19 diagnostic reagent can be used to detect metastasis in patients that suffer from gastric carcinoma.

TABLE 3
Semi-quantitative Analysis of PCR Products from Patients with
Gastric Carcinoma with and without Metastasis (Mean ± SD)

| Group | No. of cases | No. of positive | Intensive value |
|---|---|---|---|
| Gastric Ca. (M+) | 36 | 30 | 1.94 ± 0.03* |
| Gastric Ca. (M−) | 54 | 42 | 1.28 ± 0.02 |

SD; standard deviation;
Ca: carcinoma;
M+: with Metastasis
M−: without Metastasis
*: p < 0.01

### REFERENCES

The following references are hereby expressly incorporated by reference in their entireties.

1) Ren J, Chen Z, Zhou S J, Zhang X Y, Pan BR, Fan D M. Detection of Circulating Gastric Cancer Associated Antigen MG7-Ag in Human Sera with Established Immuno-PCR Technique. Cancer 2000;88(2):280-5.

2) Kohler G, Milstein C. Continuous cultures of fused cells secreting antibody pre-defined specificity. Nature 1975;256:495-99.

3) Kaminishi M, Shimizu N, Yamaguchi H, Hashimoto M, Sakai S, Oohara T. Different carcinogenesis in the gastric remnant after gstrectomy for gastric cancer. Cancer 1996;77:1646-53.

4) Tsuchya N, Hoshino K. Diagnostic significance of fluorocytophotometric DNA ploidy analysis of gastric cancer with special reference to its comparison with clinical and histopathologic diagnosis. Nippon Geka Hokan 1990;59:116-33.

5) Tahara E. Genetic alterations in human gastrointestinal cancers. The application to molecular diagnosis. Cancer 1995;75:1410-17.

6) Yasui W, Yoshida K, Ito H, Tahara E. Molecular diagnosis of gastric cancer. Gan to Kagaku Ryoho 1991;18:7-13.

7) Chen S. Biologic and molecular genetic properties of a transplantable human primary gastric cancer in nude mice. Chung Hua Chung Liu Tsa Chih 1989;11:172-75.

8) Miwa H, Wada R, Abe H, Ohkura R, Yang S W, Watanabe H, et al. Diagnosis of gastric adenoma versus early gastric cancer by bromodeoxyuridine immunohistochemistry from gastric biopsy specimen. J Gsatroenterol Hepatol 1993;8:133-37.

9) Fan DM, Zhang X Y, Chen X T, Mu Z X, Hu J L, Qioa T D, et al. Establishment of four monoclonal antibodies to a poorly differenciated gastric cnacer cell line MKN-46-9 and immunohistochemical study on their corresponding antigens. Chin. J Med PLA. 1988;13:12-15.

10) Sano T, Smith C, Cantor C. Immuno-PCR: Very sensitive antigen detection by means of specific antibody-DNA conjugates. Science 1992;258:120-22.

11) Zhou H, Fisher R J, Papas T S. Universal immuno-PCR for ultra-sensitive target protein detection. Nucleic Acids Res 1993;21 :6038-39.

12) Ren J, Fan D M, Zhou S J, Zhang X Y, Yang A G, Li M F, et al. Establishment of Immuno-PCR technique for the detection of tumor associated antigen MG7-Ag on the gastric cancer cell line. Chung Hua Chung Liu Tsa Chih 1994;16:147-50.

13) Ren J, Fan D M, Zhang X Y, Zhou S J, Yang A G, Hu J L, et al. Immuno-PCR, a new technology insero-diagnosis of gastric cancer. Chinese J Dig Dis 1996; 16:14-16.

14) Chang T C, Huang S H. A modified immuno-polymerase chain for the detection of beta-glucuronidases from Escherichia coli. J Immunol Methods 1997; 208:35-42.

15) McElhinny A S, Warner C M. Detection of major histocompatibility complex class I antigens on the surface of a single murine blastocyst by immuno-PCR. Biotechniques 1997; 23:660-62.

16) Baumler A J, Heffron F, Reissbrodt R. Rapid detection of Salmonella enterica with primers specific for iro B. J Clin Microbiol 1997; 35:1224-30.

17) Numata Y, Matsumoto Y. Rapid detection of alpha-human atrial natriuretic peptide in plasma by a sensitive immuno-PCR sandwich assays. Clin Chim Acta 1997; 259:169-76.

18) Niemeyer cM, Adler M, Blohm D. Fluorometric polymerase chain reaction (PCR) enzyme-linked immunosorbent assay for quantification of immuno-PCR products in microplates. Annl Biochem 1997; 246:140-45.

19) Suzuki, A., Itoh, F., Hinoda, Y. and Imai, K. Doudle determinant immuno-polymerase chainreaction: a sensitive method for detection circulating antigens in human sera. Jpn J Cancer Res 1995;86:885-9,

20) Sperl J, Paliwal V, Ramabhadran R, Nowak B, Askenase P W. Soluble T cell receptors: detection and quantitative assay in fluid phase via ELISA or immuno-PCR. J Immuno Methods 1995; 186:181-94.

21) Ren J, Fan D M, Zhou S J, Fan D M, Chen Z, Zhang X Y. Application of Immuno-PCR in the serologicaldiagnosis of colonic cancer. Chinese J Intern Med 1996; 35:824-25.

22) Ren J, Fan D M, Zhou S J, Zhang X Y, Yang A G, Chen S M, et al. Molecular cloning of VH, Vkgenes and sequencing of Vk genes from monclonal antibosy Mgd-1 againstric cancer. J Gastroenterol Hepatol 1993; (suppl): 226.

23) Lacey E, Grant W. photobiotin as a sensitive probe for protein labeling. Anal Biochem 1987; 163: 151-58.

24) Forster A C, McInnes J L, Skingle D C, Symons R H. Non-radioactive hybridization probes prepared by the chemical labeling of DNA and RNA with a novel reagent, photobiotin. Nucleic Acid Res 1985; 13:745-61.

25) Chen L J, Zhang S Y, Zhang J L, Zhang Z, Li S, Chen M, et al. Effect of targeting treatment of mitomycin C immunoconjugate on stomach neoplasm. Chung Kuo Yao Li Hsueh Pao 1993; 14:572-76.

26) Li S, Zhang X Y, Qiao T D, Chen X T, Zhang X Y, Chen L Y. Enhanced antitumor activity of daunomycin conjugated with antigastric cancer monoclonal antibody MGb2. Oncol Res 1992; 4:447-53.

27) Li S, Zhang X Y, Fan D M, Xhan S Y, Chen L J, et al. Specific targeting of mitomycin C to tumors by anti-gastric cancer monoclonal antibody. Chin Med J Engl 1991; 104:358-62.

28) Xu L, Zhang X Y, Zhang S Y. In vitro and on vivo targeting therapy of immunoliposomes against human gastric cancer. Chuang Hua I Hsueh Tsa Chil 1994;72:83-86.

29) Xu L. Boron neutron capture therapy of human gastric cancer by boron-containing immunoliposomes under thder thermal neutron irradiation. Chung Hua I Hsueh Tsa Chil-Taipei 1991; 71:568-71.

30) Li G, Zhang X Y, Fan D M, Hu J L. Detection of antigen-specific immune complex in sera of gastric and esophageal cancer patiens. Chin Med J Engl 1994; 197:189-91.

Although the invention has been described with reference to embodiments and examples, it should be understood that various modifications can be made without departing from the spirit of the invention. Accordingly, the invention is limited only by the following claims. All references cited herein are hereby expressly incorporated by reference in their entireties.

What is claimed is:

1. A diagnostic reagent comprising an MG7 antibody or fragment thereof joined to an avidin linker joined to a DNA having at least 50 nucleotides in length.

2. The diagnostic reagent of claim 1, wherein the DNA is at least 200 nucleotides in length.

3. The diagnostic reagent of claim 1, wherein the DNA is at least 500 nucleotides in length.

4. The diagnostic reagent of claim 1, wherein the DNA is pXJ 19.

5. The diagnostic reagent of claim 1, wherein the avidin linker is an avidin resin.

6. A biological complex comprising the diagnostic reagent of claim 1 joined to a tumor associated antigen (TAA).

7. A method of identifying the presence or absence of a tumor associated antigen (TAA) in a biological sample from an animal comprising:
obtaining a biological sample from the animal;
contacting the biological sample with a biotinylated antibody that specifically detects the tumor associated antigen so as to form an antibody/antigen complex;
contacting the antigen/antibody complex with an avidin linker so as to form an antigen/antibody/avidin linker complex;
contacting the antigen/antibody/avidin linker complex with a biotinylated DNA so as to form an antigen/antibody/avidin linker/DNA complex;
performing a Polymerase Chain Reaction (PCR) in the presence of the antigen/antibody/avidin linker/DNA complex; and
detecting the presence or absence of a PCR extension product that corresponds to the biotinylated DNA whereby the presence or absence of the TAA in the biological sample is identified.

8. The method of claim 7, wherein the TAA is associated with gastric carcinoma.

9. The method of claim 7, wherein the TAA is associated with a cancer selected from the group consisting of esophageal carcinoma, colon carcinoma, liver carcinoma, ovary carcinoma, uterine carcinoma, and lung carcinoma.

10. The method of claim 7, wherein the antibody is MG7.

11. The method of claim 7, wherein the DNA is pXJ 19.

12. The method of claim 7, wherein the animal is a human.

13. The method of claim 7, wherein the biological sample is human sera.

14. A method of identifying the presence or absence of a tumor associated antigen (TAA) in a biological sample from an animal comprising:
obtaining a biological sample from the animal;
contacting the biological sample with a diagnostic reagent comprising an antibody that specifically detects the tumor associated antigen, an avidin linker, and a biotinylated DNA so as form an antigen/diagnostic reagent complex;
performing a Polymerase Chain Reaction (PCR) in the presence of the antigen/diagnostic reagent complex; and
detecting the presence or absence of a PCR extension product that corresponds to the biotinylated DNA whereby the presence or absence of the TAA in the biological sample is identified.

15. The method of claim 14, wherein the TAA is associated with gastric carcinoma.

16. The method of claim 14, wherein the TAA is associated with a cancer selected from the group consisting of esophageal carcinoma, colon carcinoma, liver carcinoma, ovary carcinoma, uterine carcinoma, and lung carcinoma.

17. The method of claim 14, wherein the antibody is MG7.

18. The method of claim 14, wherein the DNA is pXJ 19.

19. The method of claim 14, wherein the animal is a human.

20. The method of claim 14, wherein the biological sample is human sera.

21. A method of identifying the presence or absence of metastasis in an animal comprising:
(a) obtaining a biological sample from the animal;
(b) contacting the biological sample with a biotinylated antibody that specifically detects a tumor associated antigen (TAA) so as to form an antibody/antigen complex;
(c) contacting the antigen/antibody complex with an avidin linker so as to form an antigen/antibody/avidin linker complex;
(d) contacting the antigen/antibody/avidin linker complex with a biotinylated DNA so as to form an antigen/antibody/avidin linker/DNA complex;
(e) performing a Polymerase Chain Reaction (PCR) in the presence of the antigen/antibody/avidin linker/DNA complex;
(f) determining the amount of PCR extension product that corresponds to the biotinylated DNA, whereby the amount of TAA in the biological sample is determined; and
(g) comparing the amount of TAA determined in step (f) with a standard, the amount of TAA in a biological sample obtained from an animal that has metastasis or the amount of TAA in a biological sample obtained form an animal that has cancer but does not have metastasis, whereby the presence or absence of metastasis in the animal is identified.

22. The method of claim 21, wherein the antibody is MG7.

23. The method of claim 21, wherein the DNA is pXJ 19.

24. The method of claim 21, wherein the animal is a human.

25. The method of claim 21, wherein the biological sample is human sera.

26. A method of identifying the presence or absence of metastasis in an animal comprising:
(a) obtaining a biological sample from the animal;
(b) contacting the biological sample with a diagnostic reagent comprising a biotinylated antibody that specifically detects a tumor associated antigen (TAA), an avidin linker, and a biotinylated DNA so as to form an antigen/diagnostic reagent complex;
(c) performing a Polymerase Chain Reaction (PCR) in the presence of the antigen/diagnostic reagent complex;
(d) determining the amount of PCR extension product that corresponds to the biotinylated DNA, whereby the amount of TAA in the biological sample is determined; and
(g) comparing the amount of TAA determined in step (d) with a standard, the amount of TAA in a biological sample obtained from an animal that has metastasis or the amount of TAA in a biological sample obtained form an animal that has cancer but does not have metastasis, whereby the presence or absence of metastasis in the animal is identified.

27. The method of claim 26, wherein the antibody is MG7.

28. The method of claim 26, wherein the DNA is pXJ 19.

29. The method of claim 26, wherein the animal is a human.

30. The method of claim 26, wherein the biological sample is human sera.

31. A kit comprising the diagnostic reagent of claim 1.

32. The kit of claim 31, further comprising the primers of SEQ. ID. No. 1 and 2.
